(19) United States
(12) Patent Application Publication
(10) Pub. No.: US 2021/0154167 A1
(43) Pub. Date: May 27, 2021

(54) WATER SOLUBLE FORMULATION FOR PHARMACEUTICAL AND NUTRACEUTICAL COMPOUNDS AND METHOD OF PREPARING THEREOF

(71) Applicant: HOLISTA BIOTECH SDN BHD, PETALING JAYA (MY)

(72) Inventors: RAJENDRAN MARNICKAVASAGAR, Petaling Jaya (MY); SWANAND MALODE, MUMBAI (IN); ROSCOE MICHAEL MOORE, JR., ROCKVILLE, MD (US)

(21) Appl. No.: 16/694,197

(22) Filed: Nov. 25, 2019

(51) Int. Cl. A61K 31/352; A23L 33/10; A61K 31/05; A61K 47/10; A61K 47/38; A61K 47/20; A61K 47/32; A61K 47/26; A61K 31/12

(52) U.S. Cl. CPC A61K 31/352; A23L 33/10; A61K 31/05; A61K 47/10; A61K 31/12; A61K 47/20; A61K 47/32; A61K 47/26; A61K 47/38

(57) ABSTRACT

The present invention relates to a water soluble formulation for pharmaceutical and nutraceutical compound and the method of producing thereof. The water soluble formulation of the present invention is prepared using water insoluble pharmaceutical and nutraceutical compounds such as curcuminoids and cannabinoids. The water soluble formulation of water insoluble pharmaceutical and nutraceutical compounds comprising of (i) water insoluble pharmaceutical and nutraceutical compound, (ii) surfactant, (iii) carrier and (iv) hydrophilic polymer.

### FIELD OF THE INVENTION

The present invention relates to a water soluble formulation for pharmaceutical and nutraceutical compounds and the method of producing thereof. The water soluble formulation of the present invention comprises of (i) water insoluble pharmaceutical and nutraceutical compound, (ii) surfactant, (iii) carrier and (iv) hydrophilic polymer.

### BACKGROUND OF THE INVENTION

The number of drug molecules with greater lipophilicity, larger molecular weight and poor water solubility has increased over the last few decades due to the emerging trends in combinatorial chemistry and drug design. The majority of failures in new drug development have been attributed to poor water solubility of the drug. Low-solubility drugs often show poor bioavailability. Increasing the bioavailability of low-solubility drugs has been the subject of much research.

Commonly used approaches for enhancing the dissolution rate of these molecules include nanoparticle-based formulations, lipid-based drug delivery systems, pro-drugs, amorphous solid dispersions, salt formation, co-crystals and cyclodextrin complexes.

A problem limiting the exploitation of a new drug which has potentially valuable therapeutic effects is its low bioavailability. In practice, only very low levels of active ingredient of the water insoluble compound can be absorbed in blood and tissues by oral administration. The low bioavailability of any water insoluble compound has so far limited its clinical use for prevention, and possible treatment, of diseases.

WO2014/111956 discloses a sustained release curcuminoid composition comprising a bioavailable curcuminoid composition and a release rate controlling excipient.

CN101361713 B discloses an invention relating to a curcuminoid nano crystal formulation and a preparation thereof. The nano crystal can increase the solubility of the curcuminoid, promote the absorption thereof in gastrointestinal tract and improve bioavailability.

KR101736098 B1 relates to a water-soluble resveratrol and the method of manufacturing thereof. The water-soluble resveratrol can be used for medical uses and to manufacture of a cosmetic product.

In view of the above, it appears that the low bioavailability of water insoluble pharmaceutical and nutraceutical compound hinders the therapeutic effects. There is a need to enhance water solubility of the water insoluble pharmaceutical and nutraceutical compound which in turn increases the bio-availability of the compound. This will aid to bring the water insoluble compound and its derivatives to the forefront of therapeutic agents for treatment of various diseases.

### SUMMARY OF THE INVENTION

Present invention relates to water soluble formulation of water insoluble pharmaceutical and nutraceutical compound comprises (i) water insoluble pharmaceutical and nutraceutical compound, (ii) surfactant, (iii) carrier and (iv) hydrophilic polymer. The water insoluble pharmaceutical and nutraceutical compound is selected from a group consisting of curcuminoids and cannabinoids. The surfactant is selected from a group consisting of polyethylene glycol 6000 (PEG 6000), sodium lauryl sulfate, polysorbate 80 (Tween 80) or mixture thereof. The carrier is selected from a group consisting of purified water, propylene glycol or mixture thereof. The hydrophilic polymer is selected from a group consisting of hydroxy propyl methyl cellulose, polyvinyl pyrollidone (PVP K30), copovidone (vinylpyrrolidone-vinyl acetate copolymer) or mixture thereof.

### BRIEF DESCRIPTION OF THE ACCOMPANYING DRAWINGS

The present invention will be fully understood from the detailed description given herein below and the accompanying drawings which are given by way of illustration only, and thus are not limitative of the present invention, wherein:

In the appended drawings:

FIG. 1 is a flow diagram illustrating the method used for the preparation of a water soluble solution using water insoluble pharmaceutical and nutraceutical compound in accordance to the present invention.

FIG. 2 is a chromatograph of water soluble curcuminoids obtained using turmeric powder.

FIG. 3 is a graphical representation of solubility of water soluble turmeric liquid (WSTL), water soluble turmeric powder (WSTP), curcumin 95% and turmeric powder.

FIG. 4 is a chromatograph of water soluble cannabis obtained using cannabinoids.

### DETAILED DESCRIPTION OF THE INVENTION

Detailed description of preferred embodiments of the present invention is disclosed herein. It should be understood, however, that the embodiments are merely exemplary of the present invention, which may be embodied in various forms. Therefore, the details disclosed herein are not to be interpreted as limiting, but merely as the basis for the claims and for teaching one skilled in the art of the invention. The numerical data or ranges used in the specification are not to be construed as limiting. The following detailed description of the preferred embodiments will now be described in accordance with the attached drawings, either individually or in combination.

As used herein, the term water insoluble pharmaceutical and nutraceutical compounds refer to compounds that do not dissolve in water but form sediments in the solution. Curcuminoids and cannabinoids are presented as representative examples of a water insoluble pharmaceutical and nutraceutical compound that could be prepared using the process described in the present invention.

The present invention relates to a water soluble formulation and the method of producing thereof. The water soluble formulation of the present invention is prepared using water insoluble compounds such as curcuminoids and cannabinoids.

An objective of the present invention is to provide a water soluble formulation using water insoluble pharmaceutical and nutraceutical compounds, preferably in aqueous form to improve bioavailability of water insoluble pharmaceutical and nutraceutical compounds.

Another objective of the present invention is to provide a water soluble formulation of water insoluble pharmaceutical and nutraceutical compounds using excipients which are generally recognized as safe (GRAS) to ensure safety for human consumption.

Another objective of the present invention is to provide a water soluble formulation of water insoluble pharmaceutical and nutraceutical compounds and method to produce thereof.

Yet another objective of the present invention is to provide a water soluble formulation of water insoluble pharmaceutical and nutraceutical compounds in aqueous form by using simple process to reduce the cost and time of the process.

Further objective of the present invention is to provide a taste masked water soluble formulation of water insoluble pharmaceutical and nutraceutical compounds for easy consumption.

The present invention provides a water soluble formulation of water insoluble pharmaceutical and nutraceutical compounds comprising of (i) water insoluble pharmaceutical and nutraceutical compound, (ii) surfactant, (iii) carrier and (iv) hydrophilic polymer.

The water insoluble pharmaceutical and nutraceutical compound is selected from a group consisting of curcuminoids or cannabinoids or mixtures thereof preferably curcuminoids in an amount of 0.8% to 1.0%, by weight preferably 0.9%.

The surfactant is selected from a group consisting of polyethylene glycol 6000 (PEG 6000), sodium lauryl sulfate, polysorbate 80 (Tween 80) or mixture thereof preferably polyethylene glycol 6000 in an amount of 5.0% to 6.0%, by weight preferably 5.5%.

The carrier is selected from a group consisting of water, propylene glycol or mixture thereof preferably propylene glycol in an amount of 0.75% to 1.00%, by weight preferably 0.8%.

The hydrophilic polymer is selected from a group consisting of hydroxy propyl methyl cellulose, polyvinyl pyrollidone (PVP K30), copovidone (vinylpyrrolidone-vinyl acetate copolymer) or mixture thereof preferably polyvinylpyrollidone (PVP K 30) in amount of 1.0% to 2.0%, by weight preferably 1.5%.

An exemplary embodiment for the composition for the manufacture of water soluble formulation of curcuminoids is represented in Table 1 below:

TABLE 1
Formulation of the water soluble solution of the present invention

| Material | Working range (%) | Typical range (%) |
|---|---|---|
| Water insoluble | 0.8 to 1.0 | 0.9 |
| pharmaceutical and | | |
| nutraceutical compound | | |
| Surfactant | 5.0 to 6.0 | 5.5 |
| Carrier | 0.75 to 1.0 | 0.8 |
| Hydrophilic polymer | 1.0 to 2.0 | 1.5 |

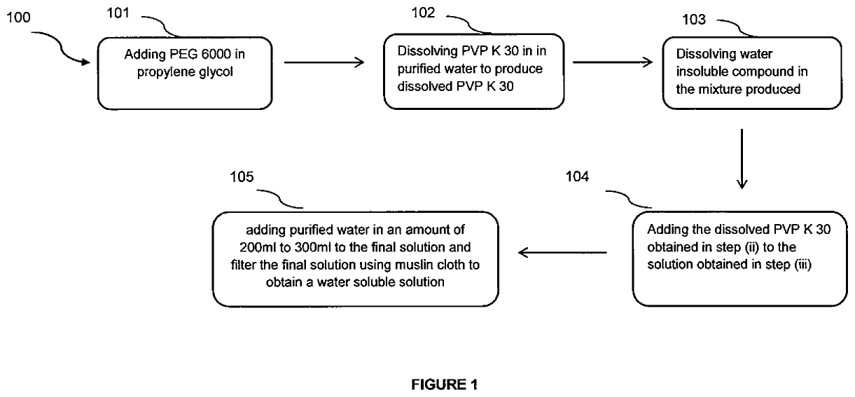

FIG. 1 is a flow diagram 100, illustrating the method used for the preparation of a water soluble solution using water insoluble compound in accordance to the present invention. The method is described in detail below.

The method of preparing a water soluble solution using water insoluble compound includes the steps of:
i. adding polyethylene glycol 6000 (PEG 6000) in an amount of 5.0% to 6.0% by weight into propylene glycol in an amount of 0.75% to 1.0% by weight to produce a mixture, (101);
ii. dissolving polyvinylpyrollidone (PVP K 30) in an amount of 1.0% to 2.0% by weight in purified water in an amount of 100 ml to 200 ml to produce dissolved PVP K 30, (102);
iii. dissolving water insoluble pharmaceutical and nutraceutical compound in an amount of 0.8% to 1.0% by weight in the mixture produced in step (i) by stirring the mixture at a temperature range of 25° C. to 30° C. to produce a solution, (103);
iv. adding the dissolved PVP K 30 obtained in step (ii) to the solution obtained in step (iii) to obtain a final solution, (104); and
v. adding purified water in an amount of 200 ml to 300 ml to the final solution and filter the final solution using muslin cloth to obtain a water soluble solution, (105).

The water soluble solution prepared in accordance to the present invention is in the form of viscous liquid. It can be easily soluble in various types of products such as aqueous based beverages, foods, pharmaceutical or nutraceutical and cosmetics products.

The water soluble solution prepared in accordance to the present invention is in aqueous form. This provides a clear solution in water with almost nil undissolved particles settled at the bottom of the solution.

The method employed in the present invention for the preparation of the water soluble solution assist to transform crystalline form of water insoluble compound to amorphous form which is soluble in water and form clear solution in the water.

An exemplary of present invention shows the chromatographic results for water soluble curcuminoids and water soluble cannabinoids of present invention.

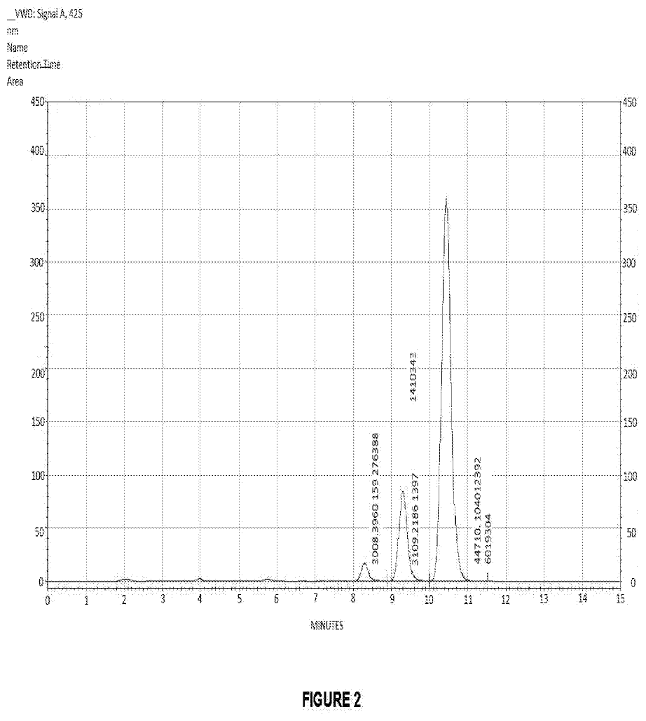

FIG. 2 is a chromatograph of water soluble curcuminoids obtained using turmeric powder. Upon experimentation it was found that the water soluble curcuminoid's of present invention retained their therapeutic properties as to the native curcuminoids.

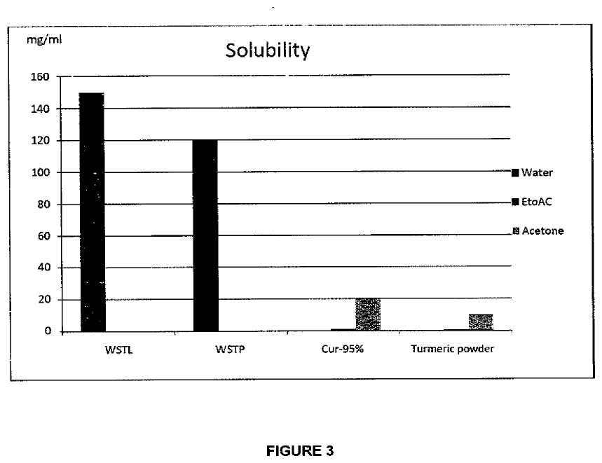

FIG. 3 is a graphical representation of solubility of water soluble turmeric liquid (WSTL), water soluble turmeric powder (WSTP), curcumin 95% and turmeric powder. Upon experimentation it was found that the WSTL has highest solubility in water.

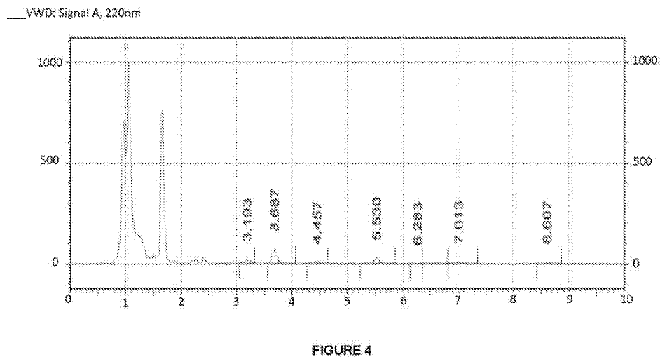

FIG. 4 is a chromatograph of water soluble cannabinoids obtained using cannabis. Upon experimentation it was found that the water soluble cannabinoids of present invention retained their therapeutic properties as to the native cannabinoids.

The water soluble formulation of water insoluble compounds with improved bioavailability is appropriate for pharmaceutical and nutraceutical compositions.

The water soluble formulation of water insoluble compounds of present invention has increased bioavailability. This leads to faster action of the drug at the site of action thus leading to more effective treatment for specific medical condition. Further, the higher solubility leads to clarity in the solution when mixed with water. This eliminates turbidity and leads to usage of the water soluble formulation of water insoluble compounds in food processing industries like beverages, aqueous based foods and the like without limiting the scope of the present invention.

Although the present invention has been described in terms of certain preferred embodiments and illustrations thereof, other embodiments and modifications to preferred embodiments may be possible that are within the principles and spirit of the invention. The above descriptions and figures are therefore to be regarded as illustrative and not restrictive.

Thus the scope of the present disclosure is defined by the appended claims and includes both combinations and sub combinations of the various features described herein above as well as variations and modifications thereof, which would occur to persons skilled in the art upon reading the foregoing description.

By using the water soluble solution prepared in the present invention, the bio-availability of water insoluble compound increases. This aids to bring water insoluble compound and its derivatives to the forefront of therapeutic agents for treatment of various diseases.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises”, “comprising”, “including”, and “having” are inclusive and therefore specify the presence of stated features, integers, steps, operations, elements, and/or components, but do not preclude the presence or addition of one or more other features, integers, steps, operations, elements, components, and/or groups thereof.

The method steps, processes and operations described herein are not to be construed as necessarily requiring their performance in the particular order discussed or illustrated, unless specifically identified as an order of performance. It is also to be understood that additional or alternative steps may be employed. The use of the expression “at least” or “at least one” suggests the use of one or more elements, as the use may be in one of the embodiments to achieve one or more of the desired objects or results.

1. A water soluble formulation for pharmaceutical and nutraceutical compound comprising of (i) water insoluble pharmaceutical and nutraceutical compound, (ii) surfactant, (iii) carrier and (iv) hydrophilic polymer.

2. The water soluble formulation for pharmaceutical and nutraceutical compound as claimed in claim 1 wherein the water insoluble pharmaceutical and nutraceutical compound is selected from a group consisting of curcuminoids and cannabinoids.

3. The water soluble formulation for pharmaceutical and nutraceutical compound as claimed in claim 1 wherein the water insoluble compound is in an amount of 0.8% to 1.0% by weight.

4. The water soluble formulation for pharmaceutical and nutraceutical compound as claimed in claim 1 wherein the surfactant is selected from a group consisting of polyethylene glycol 6000 (PEG 6000), sodium lauryl sulfate, polysorbate 80 (Tween 80) or mixture thereof.

5. The water soluble formulation for pharmaceutical and nutraceutical compound as claimed in claim 1 wherein the surfactant is in an amount of 5.0% to 6.0% by weight.

6. The water soluble formulation for pharmaceutical and nutraceutical compound as claimed in claim 1 wherein the carrier is selected from a group consisting of purified water, propylene glycol or mixture thereof.

7. The water soluble formulation for pharmaceutical and nutraceutical compound as claimed in claim 1 wherein the carrier is in an amount of 0.75% to 1.00% by weight.

8. The water soluble formulation for pharmaceutical and nutraceutical compound as claimed in claim 1 wherein the hydrophilic polymer is selected from a group consisting of hydroxy propyl methyl cellulose, polyvinyl pyrollidone (PVP K30), copovidone (vinylpyrrolidone-vinyl acetate copolymer) or mixture thereof.

9. The water soluble formulation for pharmaceutical and nutraceutical compound as claimed in claim 1 wherein the hydrophilic polymer is in amount of 1.0% to 2.0% by weight.
